(19) United States
(12) Patent Application Publication
(10) Pub. No.: US 2022/0054810 A1
(43) Pub. Date: Feb. 24, 2022

(54) Complex Aqua Skin Peeling Machine

(71) Applicant: Eunsung Global Corp., Wonju-si, Gangwon-do (KR)

(72) Inventor: Ki Se Lee, Seoul (KR)

(21) Appl. No.: 17/040,406

(22) PCT Filed: Aug. 18, 2020
(86) PCT No.: PCT/KR2020/010996

§ 371 Date: Sep. 22, 2020

(51) Int. Cl. A61M 35/00; A61M 1/00; A61K 8/19; A61Q 19/10; A61Q 19/02; A61Q 19/00; A45D 34/04

(52) U.S. Cl. CPC A61M 35/00; A61M 1/774; A61M 1/60; A61M 1/741; A61K 8/19; A61Q 19/10; A61M 2205/123; A61Q 19/008; A45D 34/04; A61M 2205/505; A61M 2210/04; A61M 2205/054; A61Q 19/02

(57) ABSTRACT

Proposed is a complex aqua skin peeling machine which allows for the use of a handpiece to supply hydrogen water to the skin of a user for removal of reactive oxygen species in the user's body.

### TECHNICAL FIELD

The present disclosure relates to a skin peeling machine and, more particularly, to a complex aqua skin peeling machine which allows for the use of a handpiece to supply hydrogen water to the skin of a user for removal of reactive oxygen species in the user's body.

### BACKGROUND ART

Unless otherwise indicated herein, the approaches described in this section are not teachings or suggestions of the prior art to the claims in this application and are not admitted to be prior art by inclusion in this section.

In general, reactive oxygen species (ROS) attack cells or genes (DNA) in the human body due to strong oxidation, which can lead to destroying the genetic information in DNA, destroying cell membranes, and forming abnormal cellular proteins, thereby providing the cause of disease and aging.

Normally, hydrogen refers to molecular hydrogen, that is, ‘H₂’, whereas active hydrogen has a strong reduction function due to the functional properties of hydrogen. Active hydrogen is significantly effective in preventing oxidative damage to DNA due to ROS, and in removing ROS generated in the skin due to ultraviolet rays or the like.

An aqua peeling apparatus is disclosed in Korean Patent No. 10-1880021.

### DISCLOSURE

### Technical Problem

An embodiment of the present disclosure relates to a complex aqua skin peeling machine which allows for the use of a handpiece to supply hydrogen water to the skin of a user for removal of reactive oxygen species in the user's body thereby preventing skin aging.

Another embodiment of the present disclosure relates to a complex aqua skin peeling machine whereby a handpiece is configured so as to be attachable to and detachable from a main body thereby increasing convenience of the procedure.

Still another embodiment of the present disclosure relates to a complex aqua skin peeling machine whereby a user is allowed to visually determine various types of treatments and level steps through a liquid crystal display (LCD) screen.

Yet another embodiment of the present disclosure relates to a complex aqua skin peeling machine whereby skin impurities, e.g., dead skin cells, are removed by means of vacuum suction.

### Technical Solution

According to an embodiment of the present disclosure in order to achieve the above and other objectives of the present disclosure,

there is provided a complex aqua skin peeling machine, including: a main body to which one or more solution containers containing functional cosmetic solutions of different ingredients are detachably coupled;

a hydrogen water container coupled to the main body, and to which a hydrogen water module that generates hydrogen water by electrolyzing water therein upon application of electric power is coupled at a lower end thereof;

an aqua handpiece including a connector portion connected to a first connection port of the main body, and a head portion to which an aqua tip is replaceably coupled, the aqua tip discharging to a user's skin a functional cosmetic solution for skin cleansing or nourishing supplied from any one of the solution containers and the hydrogen water supplied from the hydrogen water container to remove reactive oxygen species in a user's body, and suctioning the functional cosmetic solution used in skin treatment and skin impurities peeled off from the skin;

a waste storage container removably mounted on a side of the main body, and storing the functional cosmetic solution used in skin treatment and the skin impurities peeled off from the skin, which are suctioned through the aqua tip; and

a touch screen controlling an operation of the hydrogen water module, controlling opening and closing of a solenoid valve that controls supply of the functional cosmetic solutions and the hydrogen water to the aqua handpiece from the solution containers and the hydrogen water container, and controlling an operation of the aqua handpiece, in response to a touch input.

### Advantageous Effects

The complex aqua skin peeling machine according to the embodiment of the present disclosure having the above-described configuration has the following advantages.

A handpiece is used to supply hydrogen water to the skin of a user for removal of reactive oxygen species in the user's body, thereby preventing skin aging by simultaneously performing the removal of the reactive oxygen species in the user's body and deep cleansing, and to supply a functional cosmetic solution to the user's skin, thereby providing a skin whitening effect.

The handpiece is configured so as to be attachable to and detachable from a holder of the main body, thereby providing convenience of the procedure to the user.

The user is allowed to visually and easily determine various types of treatments, and level steps through an LCD screen, thereby providing use convenience to the user.

Skin impurities, e.g., dead skin cells, sebum, etc., are removed by means of vacuum suction, thereby providing a peeling function.

### DESCRIPTION OF DRAWINGS

FIG. 1 is a front perspective view illustrating a complex aqua skin peeling machine according to a preferred embodiment of the present disclosure;

FIG. 2 is a rear perspective view illustrating the skin peeling machine illustrated in FIG. 1;

FIG. 3 is a schematic exploded perspective view illustrating the skin peeling machine illustrated in FIG. 1;

FIG. 4 is a front view illustrating the skin peeling machine illustrated in FIG. 1;

FIG. 5 is a side view illustrating the skin peeling machine illustrated in FIG. 1;

FIG. 6 is a view illustrating an aqua handpiece of the skin peeling machine illustrated in FIG. 1;

FIG. 7 is a sectional view illustrating the aqua handpiece illustrated in FIG. 5;

FIG. 8 is a sectional view taken along line A-A illustrated in FIG. 4;

FIG. 9 is a sectional view taken along line B-B illustrated in FIG. 4;

FIG. 10 is a partial cutaway perspective view illustrating the internal structure of the skin peeling machine illustrated in FIG. 1; and

FIG. 11 is a photographic representation of a touch screen of the skin peeling machine illustrated in FIG. 1.

### DESCRIPTION OF THE REFERENCE NUMERALS IN THE DRAWINGS

10, 11, 12; solution container

14; hydrogen water module

16; first connection port

18; aqua handpiece

20; functional handpiece

22; waste storage container

23, 24, 25, 26; solenoid valve

28; first holder

30; level sensor

32; icon

34; second control knob

36; vacuum pump

38; front cover

40; aqua tip cap

### Best Mode

Hereinafter, a complex aqua skin peeling machine according to a preferred embodiment of the present disclosure will be described in detail with reference to the accompanying drawings.

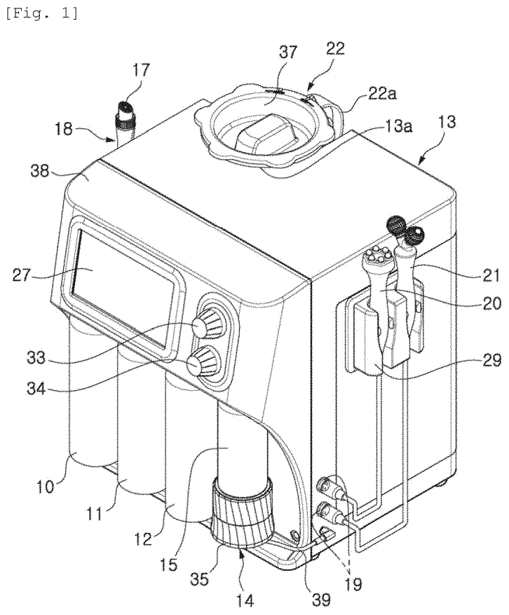

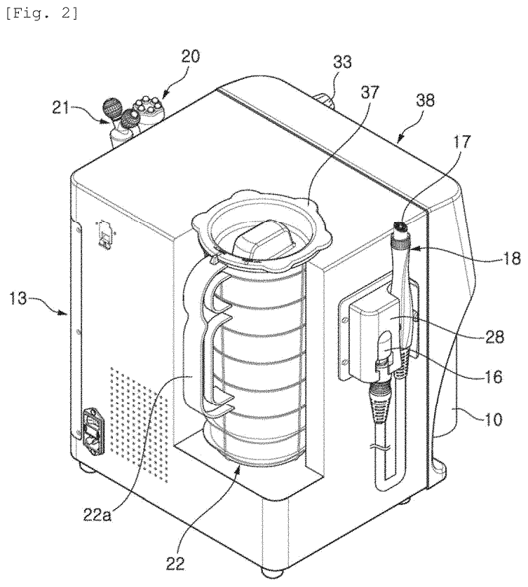

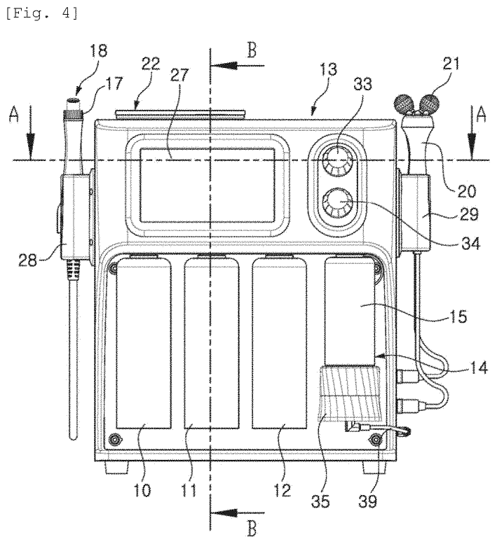

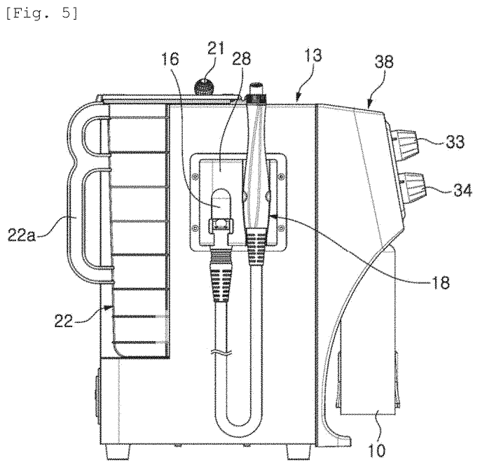

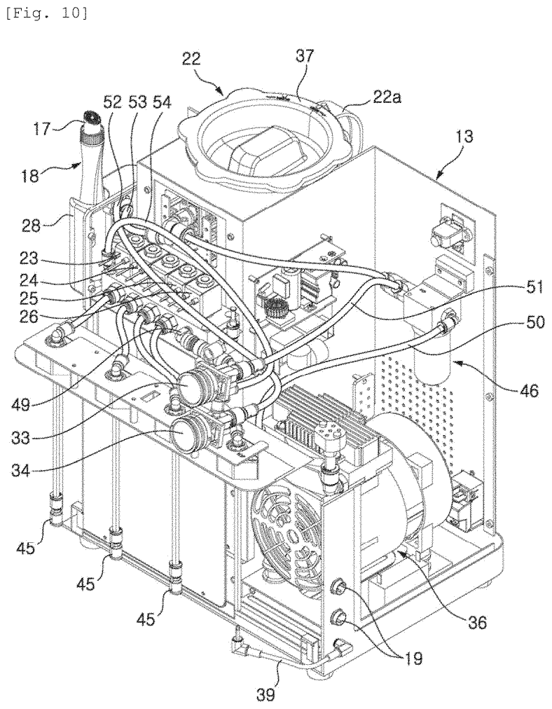

Referring to FIGS. 1 to 11, a complex aqua skin peeling machine according to an embodiment of the present disclosure includes:

a main body 13 to which one or more solution containers 10, 11, and 12 containing functional cosmetic solutions of different ingredients are detachably coupled by screw engagement;

a hydrogen water container 15 detachably coupled to the main body 13 by screw engagement, and to which a hydrogen water module 14 that generates hydrogen water by electrolyzing water therein upon application of electric power is detachably coupled at a lower end thereof by screw engagement;

an aqua handpiece 18 including a connector portion 18b detachably connected to a first connection port 16 of the main body 13, and a head portion 18a to which an aqua tip 17 is replaceably coupled, the aqua tip discharging to the user's skin a functional cosmetic solution for skin cleansing or nourishing supplied from any one of the solution containers 10, 11, and 12 and the hydrogen water supplied from the hydrogen water container 15 to remove reactive oxygen species in the user's body, and suctioning the functional cosmetic solution used in skin treatment and skin impurities peeled off from the skin;

a waste storage container 22 removably mounted on a side of the main body 13, and in which the functional cosmetic solution used in skin treatment and the skin impurities (e.g., dead skin cells, sebum, acne, etc.) peeled off from the skin, which are suctioned through the aqua tip 17 and a waste suction hose 53, are stored as a vacuum is formed therein by being connected to a vacuum pump 36 through hoses 50 and 55; and

a touch screen 27 controlling the operation of the hydrogen water module 14, opening and closing of solenoid valves 23, 24, 25, and 26 that control the supply of the functional cosmetic solutions and the hydrogen water to the aqua handpiece 18 from the solution containers 10, 11, and 12 and the hydrogen water container 15, and the operation of the aqua handpiece 18, in response to a touch input.

According to a more preferred embodiment, as illustrated in FIGS. 1, 3, and 8, the complex aqua skin peeling machine further includes one or more functional handpieces 20 and 21 connected to a second connection port 19 of the main body 13, and activating cells by massaging the skin by contracting and relaxing muscles through low- or high-frequency electrical stimulation upon application of electric power.

At this time, the functional handpieces are an electroporation handpiece 20 and a Y handpiece 21.

As illustrated in FIGS. 1, 2, 4 and 8, the aqua handpiece 18 and the functional handpieces 20 and 21 described above are detachably secured in place to a first holder 28 and a second holder 29, respectively, formed on an outer surface of the main body 13.

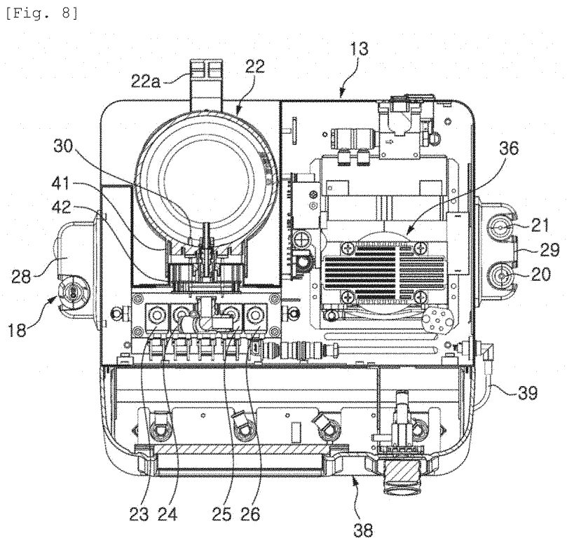

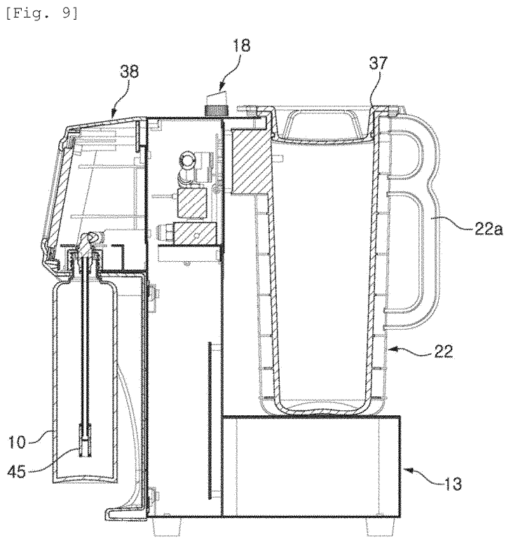

As illustrated in FIGS. 8 and 9, the above-described waste storage container 22 further includes:

a metal level sensor 30 provided at an arbitrary position on an upper portion of an inner surface of the waste storage container 22, and detecting when waste is filled in the waste storage container 22 to a predetermined arbitrary level and outputting a detection signal so that a warning is displayed on the touch screen 27 to notify the disposal time of the waste.

At this time, the technique of detecting the level of the waste by the level sensor 30 when the waste recovered after contact with the user's skin is filled in the waste storage container 22 to the arbitrary level and transmitting the detection signal to a controller (not illustrated) is commonly used in the art, and thus a detailed description thereof will be omitted.

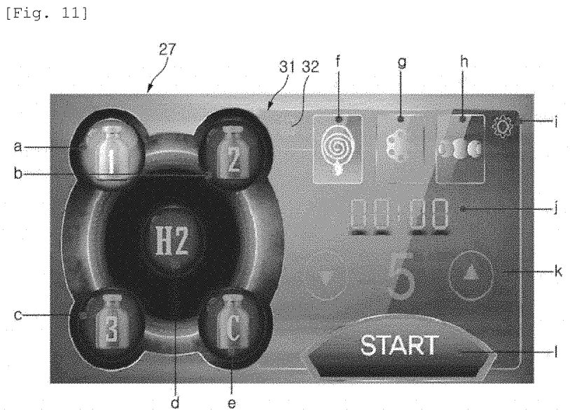

As illustrated in FIG. 11, the above-described touch screen 27 further includes

a water level warning display 31 and an icon 32 configured such that when the waste is filled in the waste storage container 22 to the predetermined arbitrary level, the water level warning display 31 displays ‘water full’ while the icon 32 flashes.

The solution containers 10, 11, and 12 and the hydrogen water container 15 described above are made of

a transparent body (e.g., a synthetic resin material is used) so that the user can visually ascertain the remaining amount of the functional cosmetic solutions and the hydrogen water from outside.

As illustrated in FIGS. 1, 4, and 5, the above-described main body 13 further includes:

a dial-type first control knob 33 variably controlling a vacuum to supply the functional cosmetic solution to the user's skin through the aqua tip 17 of the aqua handpiece 18, or to suction the functional cosmetic solution used in skin treatment and the skin impurities peeled off from the skin; and

a dial-type second control knob 34 variably controlling flows of the functional cosmetic solutions and the hydrogen water suctioned from the solution containers 10, 11, and 12 and the hydrogen water container 15 and supplied to the aqua handpiece 18.

As illustrated in FIGS. 1, 3, and 4, the complex aqua skin peeling machine further includes a non-slip 35 (e.g., a waved uneven portion) formed on an outer surface of the above-described hydrogen water module 14, and preventing the hydrogen water module 14 from slipping from the user's hand when the hydrogen water module 14 is coupled to or separated from the hydrogen water container 15.

Therefore, when the user couples the hydrogen water module 14 to the hydrogen water container 15 or separates the same therefrom by holding the outer surface of the hydrogen water module 14 with the hand, slipping of the hydrogen water module 14 from the hand of the user can be prevented by the non-slip 35, thereby providing use convenience to the user.

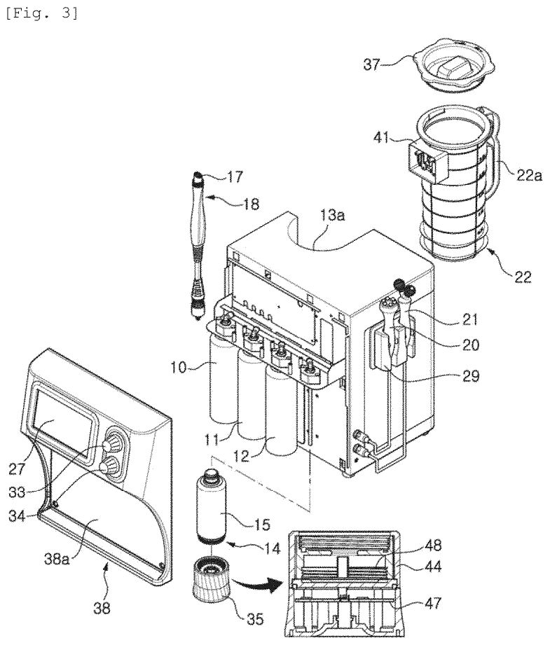

As illustrated in FIGS. 3, 8, and 9, an arc-shaped seat portion 13a corresponding to an outer circumferential surface of a cylindrical portion of the waste storage container 22 is formed in the main body 13 so that when the waste storage container 22 is mounted on the main body 13 by fitting engagement between a quadrangular female protrusion 41 formed on an outer circumferential surface of the above-described waste storage container 22 and a quadrangular male protrusion 42 formed on a rear surface of the main body 13 corresponding thereto, the waste storage container 22 is brought into close contact with the cylindrical portion of the waste storage container 22 so as to minimize protrusion from a receiving part of the main body 13.

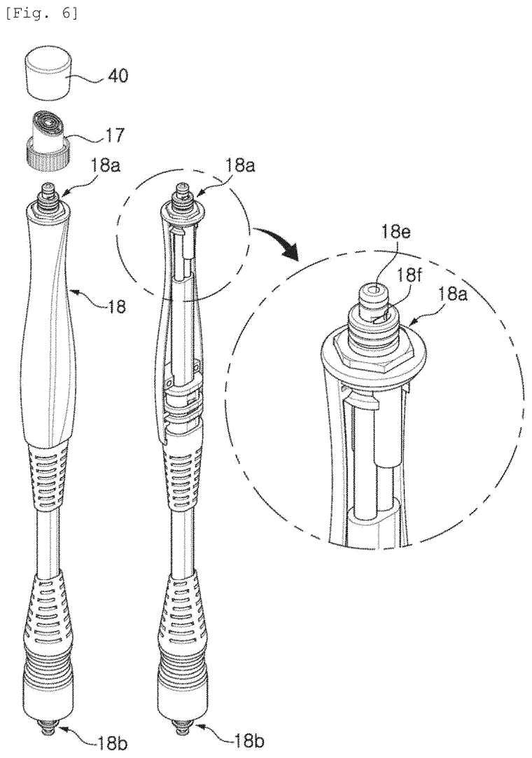

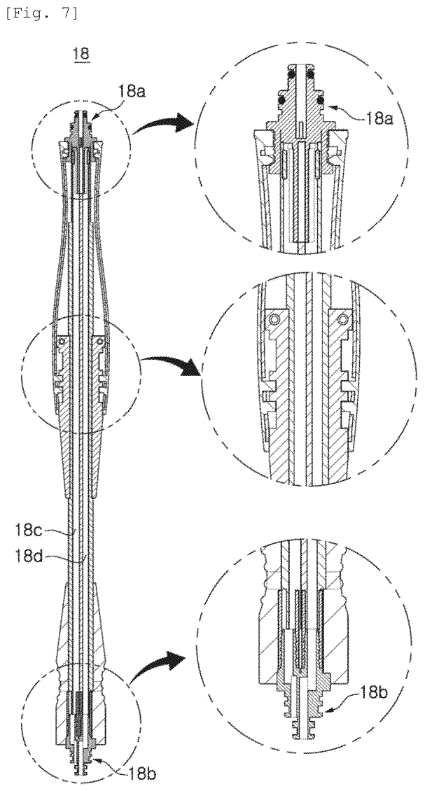

As illustrated in FIGS. 6 and 7, the above-described aqua handpiece 18 includes: the head portion 18a discharging to the user's skin the functional cosmetic solutions supplied from the solution containers 10, 11, and 12 through a supply tube 18c and a cosmetic solution discharge hole 18c or the hydrogen water supplied from the hydrogen water container 15 through the supply tube 18c, or suctioning the functional cosmetic solution used in skin treatment and the skin impurities peeled off from the skin through a waste suction hole 18f and discharging the suctioned cosmetic solution and skin impurities through a waste discharge tube 18d; and the connector portion 18b detachably connected to the first connection port 16 of the main body 13.

As illustrated in FIG. 6, the aqua tip 17 that is replaceably coupled to the above-described head portion 18a is operated in such a manner that as a vacuum is formed in a portion of the aqua tip 17 brought into contact with the user's skin, the skin impurities peeled off from the skin are suctioned through the waste suction hole 18f of the head portion 18a communicating with a waste suction hole (not designated by reference numeral) formed at a peripheral portion of the aqua tip 17; and the cosmetic solution is applied to the skin through the cosmetic solution discharge hole 18e of the head portion 18a communicating with a cosmetic solution discharge hole (not designated by reference numeral) formed at a central portion of the aqua tip 17.

Therefore, the aqua tip 17, which is replaceably coupled to the head portion 18a, is used for skin care and removal of reactive oxygen species in the user's body by supplying the functional cosmetic solution or the hydrogen water upon contact with the skin of the user.

At this time, the technique of discharging the functional cosmetic solutions supplied from the solution containers 10, 11, and 12 or the hydrogen water supplied from the hydrogen water container 15 to the user's skin through the cosmetic solution discharge hole 18e of the head portion 18a connected to the supply tube 18c of the aqua handpiece 18 and through the cosmetic solution discharge hole of the aqua tip 17 and the technique of suctioning the functional cosmetic solution used in skin treatment and the skin impurities peeled off from the skin through the waste suction hole of the aqua tip 17 and through the waste suction hole 18f and the waste discharge tube 18d of the head portion 18a are commonly used in the art, and thus detailed descriptions thereof will be omitted.

The aqua tip 17 replaceably coupled to the head portion 18a of the aqua handpiece 18 may be used by selecting a large- or small-size aqua tip 17 depending on the area of the treatment sites e.g., the user's face, forehead, etc., and a portion of the aqua tip 17 brought into contact with the skin may be formed with an inclination.

Hereinafter, a use example of the complex aqua skin peeling machine according to the embodiment of the present disclosure will be described with reference to the accompanying drawings.

As illustrated in FIGS. 1 to 11, a cable of the aqua handpiece 18 is coupled to the first connection port 16 formed on the first holder 28 mounted on a first side surface of the above-described main body 13, and then is pushed upward in the drawings for connection.

At this time, the aqua handpiece 18 is mounted to the first holder 28 and secured in place, and the aqua tip 17 is detachably coupled to the end of the aqua handpiece 18. In a case where the skin peeling machine is not to be used for a long period of time, the aqua tip 17 (used for disposable use) is removed from the aqua handpiece 18, and then an aqua tip cap 40 is placed on the aqua handpiece 18 for storage.

Respective cables of the above-described functional handpieces, i.e., the Y handpiece 21 and the electroporation handpiece 20, are coupled to and connected to the second connection port 19 formed on a second side surface of the main body 13.

The solution containers 10,11, and 12 containing the functional cosmetic solutions (each of which is used by mixing solution concentrate and distilled water at an arbitrary ratio) are detachably screwed to the main body 13 through a cutout portion 38a of the front cover 38 mounted on a front surface of the above-described main body 13.

At this time, screwing the solution containers 10, 11, and 12 detachably to the above-described main body 13 has been described as an example, but is not limited thereto, and is commonly used in the art, and thus a detailed description thereof will be omitted.

A filter 45 filtering out foreign substances contained in each of the functional cosmetic solutions is mounted at an end of a hose (not designated by reference numeral) that is included in each of the above-described solution containers 10, 11, and 12 to suction the functional cosmetic solution.

The hydrogen water container 15 with the hydrogen water module 14 detachably screwed to a lower end thereof is detachably screwed to a position on the right of the solution container 12 coupled to the above-described main body 13.

As illustrated in FIGS. 1, 3, 4, 8, and 10, the functional cosmetic solutions contained in the above-described solution containers 10, 11, and 12 and the hydrogen water contained in the above-described hydrogen water container 15 are moved to a valve block 49 along respective hoses (not designated by reference numeral).

The functional cosmetic solutions or the hydrogen water moved to the valve block 49 are passed through the respective solenoid valves 23, 24, 25, and 26 that are switched to an open state upon application of an electric signal.

The functional cosmetic solutions or the hydrogen water passed through the solenoid valves 23, 24, 25, and 26 are passed through a fluid moving hose 52 and moved to the second control knob 34, so that depending on the amount of rotation of the dial-type second control knob 34, it is possible to control the flow of the functional cosmetic solutions or the hydrogen water through the fluid moving hose 52 to the aqua tip 17 coupled to the head portion 18a of the aqua handpiece 18.

Meanwhile, the functional cosmetic solution used in skin treatment and the skin impurities peeled off from the skin are suctioned through the aqua tip 17, and suctioned into the waste storage container 22 through the waste suction hose 53 by the hose 55 connected to the air filter 46 to form a vacuum inside the waste storage container 22.

At this time, a vacuum formed by the vacuum pump 36 is passed through the hose 50 and moved to the air filter 46, and the vacuum moved to the air filter 46 is passed through the hose 51 and moved to the first control knob 33. Depending on the amount of rotation of the dial-type first control knob 33, it is possible to control the vacuum that is passed through a hose 54 and moved to the valve block 49.

To apply electric power to the hydrogen water module 14, a connector (not designated by reference numeral) formed on the outer surface of the main body 13 and a connector (not illustrated) formed on a bottom surface of the hydrogen water module 14 are electrically connected to each other by a power cable 39.

Therefore, as electric power is applied to an electrode plate 48 for electrolysis through the above-described power cable 39 through a printed circuit board (PCB) 47 mounted inside a casing 44 of the hydrogen water module 14, the hydrogen water (active hydrogen water) is generated by electrolyzing water filled in the hydrogen water container 15 with a bottom surface being in communication with the hydrogen water module 14.

In addition, as the inside of the hydrogen water container 15, which is a transparent body, is irradiated with light from a light emitting diode (LED, not designated by reference numeral) mounted on the PCB 47, the user can visually ascertain that the hydrogen water is generated from outside the hydrogen water container 15.

At this time, the technique of generating the hydrogen water by electrolyzing the water filled in the hydrogen water container 15 upon application of electric power to the electrode plate 48 for electrolysis of the hydrogen water module 14 is commonly used in the art, and thus a detailed description thereof will be omitted.

The solution containers 10, 11, and 12 and the hydrogen water container 15 described above are made of a transparent body (e.g., a synthetic resin material is used) so that the user can visually ascertain the remaining amount of the functional cosmetic solutions and the hydrogen water from outside.

The waste storage container 22 is removably mounted on the rear surface of the main body 13. That is, by fitting engagement between the quadrangular female protrusion 41 formed on the outer circumferential surface of the waste storage container 22 and the quadrangular male protrusion 42 formed on the main body 13 corresponding thereto, the waste storage container 22 is mounted to the receiving part of the main body 13.

At this time, since the cylindrical waste storage container 22 is brought into close contact with the arc-shaped seat portion 13a formed in the receiving part of the main body 13, it is possible to minimize protrusion of the waste storage container 22 from the receiving part of the main body 13.

When the functional cosmetic solution used in skin treatment of the user and the skin impurities (dead skin cells, sebum, acne, etc.) peeled off from the skin continue to be suctioned into the waste storage container 22 through the aqua handpiece 18 and are filled in the waste storage container 22 to the predetermined arbitrary level, this is detected by the metal level sensor 30 mounted inside the waste storage container 22.

That is, when the disposal time of the waste filled in the waste material storage container 22 is detected by the level sensor 30, the water level warning display 31 of the touch screen 27 displays ‘water full’ while the icon 32 flashes repeatedly. At this time, the operation of the skin peeling machine is automatically stopped by a program stored in the controller.

Therefore, after the waste storage container 22 is removed and separated from the above-described main body 13 by holding a handle 22a of the waste storage container 22, a lid 37 is opened so that the waste can be discharged to a designated place.

As illustrated in FIG. 11, as electric power is applied to the above-described main body 13, a main screen of the touch screen 27 is loaded (see FIG. 11). When touching a setting button i on the main screen, the user can turn button volume ON/OFF and perform screen calibration, and also can ascertain a device model name and a software version.

a) An aqua peeling mode will be described with reference to the accompanying drawings.

As illustrated in FIG. 11, when the user (administrator) touches an aqua handpiece selection button f to select the aqua peeling mode, with a desired treatment selection button (any one of a, b, c, and d) touched, when the user touches a start/stop button 1, the operation starts, and a treatment time j is displayed with the start of the operation.

That is, when the user touches the above-described treatment selection button a, a cosmetic treatment is performed by discharging a functional cosmetic solution (e.g., a cleansing liquid may be used) supplied from the solution container 10 to the user's skin through the aqua tip 17 of the aqua handpiece 18.

When the user touches the above-described treatment selection button b, a cosmetic treatment is performed by discharging a functional cosmetic solution (e.g., a deep cleansing liquid may be used) supplied from the solution container 11 to the user's skin through the aqua tip 17 of the aqua handpiece 18.

When the user touches the above-described treatment selection button c, a cosmetic treatment is performed by discharging a functional cosmetic solution (e.g., an essence liquid may be used) supplied from the solution container 12 to the user's skin through the aqua tip 17 of the aqua handpiece 18.

When the user touches the above-described treatment selection button d, a hydrogen water therapy treatment is performed by discharging the hydrogen water supplied from the hydrogen water container 15 to the user's skin through the aqua tip 17 of the aqua handpiece 18.

The user can control the appropriate pressure and discharge amount discharged through the aqua tip 17 of the aqua handpiece 18 by rotating the first control knob 33 and the second control knob 34 are rotated clockwise in the drawings.

The user brings the aqua tip 17 of the aqua handpiece 18 into precisely close contact with the user's skin thereby starting the procedure.

When the user wants to change a treatment type during the procedure, the user touches the start/stop button 1 to stop the operation, and then the user touches a desired treatment button and touches the start/stop button 1 to start the operation.

After performing Treatment 1 and Treatment 2 and before performing Treatment 3, the aqua tip 17 is immersed in water and washed by shaking, followed by vacuum suction for about 5 seconds to remove foreign substances.

When the user touches a cleansing mode button e, washing automatically proceeds for a predetermined period of time. That is, after adjusting the first control knob 33 and the second control knob 34 to the highest levels, when the user touches the start/stop button 1, a cleansing mode automatically proceeds for a predetermined period of time.

At this time, the head portion 18a of the aqua handpiece 18 has to be closed with the aqua tip cap 40.

When the user touches the hydrogen water selection button d to perform the hydrogen water therapy treatment, the hydrogen water selection button d flashes for a predetermined period of time, and the operation of increasing the hydrogen level of the hydrogen water to the maximum level starts.

When the user touches the start/stop button 1, the operation starts, and the user then brings the aqua tip 17 coupled to the aqua handpiece 18 into precisely close contact with the user's skin thereby starting the procedure.

When the procedure for a predetermined period of time is finished, the user touches the start/stop button 1 to stop the operation.

b) An electroporation mode will be described with reference to the accompanying drawings.

As illustrated in FIG. 11, the user (administrator) touches an electroporation selection button g to select the electroporation mode.

The user controls the electroporation level by using a level control button k.

When the user touches the start/stop button 1, the operation starts thereby starting skin care with the functional handpiece (electroporation handpiece) 20. At this time, performing skin care with electrical stimulation (low frequency or high frequency) using the electroporation handpiece 20 is a method commonly used in the art, and thus a detailed description thereof will be omitted.

When the user repeatedly touches the electroporation selection button g during the procedure, an LED changes in the order of red, blue, purple, and off.

When the procedure for a predetermined period of time is finished, the user touches the start/stop button 1 to stop the operation.

c) A Y handpiece mode will be described with reference to the accompanying drawings.

As illustrated in FIG. 11, the user (administrator) touches a Y handpiece selection button h to select the Y handpiece mode.

The user controls the Y handpiece level by using the level control button k.

When the user touches the start/stop button 1, the operation starts thereby starting skin care with the functional handpiece (Y handpiece) 21. At this time, performing skin care with electrical stimulation (low frequency or high frequency) using the Y handpiece 21 is a method commonly used in the art, and thus a detailed description thereof will be omitted.

When the procedure for a predetermined period of time is finished, the user touches the start/stop button 1 to stop the operation.

As described above, according to the complex aqua skin peeling machine according to the embodiment of the present disclosure,

the hydrogen water in the hydrogen water container 15 generated by the above-described hydrogen water module 14 is discharged to the user's skin through the aqua tip 17 of the aqua handpiece 18 so that active hydrogen contained in the hydrogen water is brought into contact with the skin thereby removing reactive oxygen species from the user's body, it is possible to prevent aging of the skin of the user.

In addition, by deep cleansing with the functional cosmetic solutions supplied through the aqua tip from the solution containers 10, 11, and 12, it is possible to obtain a peeling function of removing skin impurities, e.g., dead skin cells, spots, sebum, etc., and a skin whitening effect.

Although the preferred embodiment has been described for illustrative purposes in the present disclosure, those skilled in the art will appreciate that various modifications, additions and substitutions are possible, without departing from the scope and spirit of the disclosure as disclosed in the accompanying claims.

### Industrial Applicability

A complex aqua skin peeling machine according to an embodiment of the present disclosure can allow for the use of a handpiece

to supply hydrogen water to reactive oxygen species for removal of the reactive oxygen species thereby preventing skin aging, and to remove skin impurities, e.g., dead skin cells, by means of vacuum suction.

1. A complex aqua skin peeling machine, comprising:
a main body to which one or more solution containers containing functional cosmetic solutions of different ingredients are detachably coupled;
a hydrogen water container coupled to the main body, and to which a hydrogen water module that generates hydrogen water by electrolyzing water therein upon application of electric power is coupled at a lower end thereof;
an aqua handpiece including a connector portion connected to a first connection port of the main body, and a head portion to which an aqua tip is replaceably coupled, the aqua tip discharging to a user's skin a functional cosmetic solution for skin cleansing or nourishing supplied from any one of the solution containers and the hydrogen water supplied from the hydrogen water container to remove reactive oxygen species in a user's body, and suctioning the functional cosmetic solution used in skin treatment and skin impurities peeled off from the skin;
a waste storage container removably mounted on a side of the main body, and in which the functional cosmetic solution used in skin treatment and the skin impurities peeled off from the skin, which are suctioned through the aqua tip, are stored; and
a touch screen controlling an operation of the hydrogen water module, controlling opening and closing of a solenoid valve that controls supply of the functional cosmetic solutions and the hydrogen water to the aqua handpiece from the solution containers and the hydrogen water container, and controlling an operation of the aqua handpiece, in response to a touch input.

2. The complex aqua skin peeling machine of claim 1, further comprising:
at least one functional handpiece connected to a second connection port of the main body, and activating cells by massaging the skin by contracting and relaxing muscles through low- or high-frequency electrical stimulation upon application of electric power.

3. The complex aqua skin peeling machine of claim 1, wherein the aqua handpiece and the functional handpiece are detachably secured in place to a first holder and a second holder, respectively, formed on an outer surface of the main body.

4. The complex aqua skin peeling machine of claim 1, wherein the waste storage container further comprises a metal level sensor provided on an inner surface of the waste storage container, and detecting when waste is filled in the waste storage container to a predetermined arbitrary level and outputting a detection signal so that a warning is displayed on the touch screen to notify a disposal time of the waste.

5. The complex aqua skin peeling machine of claim 4, wherein the touch screen further comprises a water level warning display and an icon configured such that when the waste is filled in the waste storage container to the predetermined arbitrary level, the water level warning display displays ‘water full’ while the icon flashes.

6. The complex aqua skin peeling machine of claim 1, wherein the solution containers and the hydrogen water container are made of a transparent body so that a user can visually ascertain a remaining amount of the functional cosmetic solutions and the hydrogen water from outside.

7. The complex aqua skin peeling machine of claim 1, wherein the main body further comprises:
a dial-type first control knob variably controlling a vacuum to supply the functional cosmetic solution to the user's skin through the aqua tip of the aqua handpiece, or to suction the functional cosmetic solution used in skin treatment and the skin impurities peeled off from the skin; and
a dial-type second control knob variably controlling flows of the functional cosmetic solutions and the hydrogen water suctioned from the solution containers and the hydrogen water container and supplied to the aqua handpiece.

8. The complex aqua skin peeling machine of claim 1, further comprising:
a non-slip formed on an outer surface of the hydrogen water module, and preventing the hydrogen water module from slipping from a user's hand when the hydrogen water module is coupled to or separated from the hydrogen water container.

9. The complex aqua skin peeling machine of claim 1, wherein an arc-shaped seat portion is formed in the main body so that when the waste storage container is mounted on the main body by fitting engagement between a quadrangular female protrusion formed on an outer circumferential surface of the waste storage container and a quadrangular male protrusion formed on a rear surface of the main body corresponding thereto, the waste storage container is brought into close contact with a cylindrical portion of the waste storage container.
